(19) United States
(12) Patent Application Publication
(10) Pub. No.: US 2017/0071766 A1
(43) Pub. Date: Mar. 16, 2017

(54) COMPRESSIBLE SELF-EXPANDABLE STENT FOR SPLINTING AND/OR KEEPING OPEN A CAVITY, AN ORGAN DUCT, AND/OR A VESSEL IN THE HUMAN OR ANIMAL BODY

(71) Applicants: Klaus Düring, Frechen (DE); Nasib Dlaikan-Campos, Wuerselen (DE)

(72) Inventors: Klaus Düring, Frechen (DE); Nasib Dlaikan-Campos, Wuerselen (DE)

(21) Appl. No.: 15/125,251

(22) PCT Filed: Mar. 13, 2015
(86) PCT No.: PCT/EP2015/000557

§ 371 Date: Sep. 12, 2016

(30) Foreign Application Priority Data
Mar. 13, 2014 (DE) 10 2014 003 654.5

(51) Int. Cl. A61F 2/90; A61F 5/56

(52) U.S. Cl. CPC A61F 2/90; A61F 5/566; A61F 2250/0039

(57) ABSTRACT

A compressible, self-expandable stent for splinting and/or keeping open a cavity, an organ duct, and/or a vessel in the human or animal body is disclosed. The stent is configured in a tubular fashion and has a mesh of at least one wire. At a distal end of the stent, a portion each of the wire is bent into a loop, whereas the portion of the wire is wrapped around to form a twist tie, by means of which the loop is closed.

The present invention relates to a compressible and self-expandable stent for splinting and/or keeping open a cavity, an organ duct, and/or a vessel in the human or animal body.

Syndromes based on the at least partial closure of cavities, organ ducts and/or vessels are strongly increasing due to the effects of civilization. An important part of such syndromes relate to the air passages. Obstructive sleep apnea, for example, is a life-threatening illness resulting from the closure of the rear air passage due to muscle relaxation during sleep. Due to repeated and sometimes very frequent interruption of breathing during sleep the patient is not adequately supplied with oxygen, in extreme cases leading to death. Civilization-related factors such as e.g. severe obesity or excessive alcohol consumption seriously increase the risk of illness. Snoring is a more gentle outcome of the effect than sleep apnea but results from the same organic cause. Whereas people are aware of snoring, the serious risks to health especially of heavy snoring and of sleep apnea are often not registered, since the patient is not aware of the organic deficiency symptoms.

The breathing stops result from obstructions of the airway in the pharynx, mostly in the region of the velopharynx, partially also the hypopharynx. Suction of the soft tissue due to the created vacuum resulting from small diameters of the airway predominantly occurs in the velopharynx as there is present the largest part of pharyngeal soft tissue. Consequently, splinting of the velopharynx is a key step in sleep apnea therapy.

WO 2007/065408 A2 shows an apnea stent which serves for splinting and/or keeping open the airway in the throat. This apnea stent consists of a compressible and self-expanding stent which comprises at least one widened section. This stent is designed with three phases. The distal phase of the stent forms a functional part of the apnea stent. Therein, this part of the stent does not actively push open a lumen. Rather, during suction of the soft tissue due to the vacuum created during the apneas and hypopneas the stent prevents an obstruction of the airway in that way that the sucked-in soft tissue attaches to the stent in its expanded state. This distal phase is tubularly shaped and expandable in a way that the airway is kept open. A proximal phase of the stent is provided for fixation of the stent in the nose region. The proximal phase has a funnel-type shape, wherein it widens from a proximal to a distal end. The distal and the proximal phase are formed as a braid in which wires or fibers or filaments, resp., intersect. Each single wire running into the direction of the distal end of the stent is led back at the distal end of the distal phase towards the proximal end of the stent. The bendings created in this way are called round ends. A transition phase of the stent is provided for connecting the proximal phase with the distal phase. The transition phase is formed by twisting of the wires of the stent to result in twisted strands. Thereby, the transition phase has a higher flexibility in radial direction.

WO 2012/107229 A2 discloses a stent for splinting of a nasal passage. This stent consists of a braided tubular support body which in unloaded state has a diameter of at least 4 mm and a length in the range of 25 mm to 100 mm or 25 mm to 120 mm, resp. The stent can have a widened section at the proximal end to support the nasal alar. Alternatively, or in combination, it can be provided with a fixation section which is provided at the proximal end of the stent and intended for fixation of the stent in a nasal passage of the user. During fixation the fixation section extends to the outside of the nose of the user and can be fixed outside of the nose.

US 2004/0134491 A1 discloses an apparatus for treatment of sleep apnea. This apparatus, by its size, is adapted for being positioned in a predetermined location in the oropharyngeal region. The apparatus comprises a loop which delimits an internal space and has a first and a second end region. The loop is defined by two elongated elements spaced from one another and which extend from the first to the second end region, whereby the apparatus expands in a C-shaped design in which the elongated elements press against the lateral walls of the oropharyngeal region and in this way exert an opening force in this region.

US 2001/0044647 A1 discloses a modular endoluminal stent graft. This stent graft comprises at least two stent grafts of different sizes, whereby one stent graft is deployed inside the other one. The first stent graft provides a chronically widened end which is expandable to a first diameter, and a middle section which is expandable to a second diameter being smaller than the first diameter. The second stent graft provides an end which is expandable to a diameter fitting into a middle section of the first stent graft.

US 2003/0153973 A1 discloses a braided modular stent with hourglass-shaped interfaces. This stent comprises a first component and a second component wherein each component has an hourglass-shaped interface. It is foreseen that a section with a reduced diameter has a higher radial stiffness than sections with nominal diameter whereas the section with the reduced diameter of the hourglass-shaped interface has a larger braiding angle than the sections with nominal diameter.

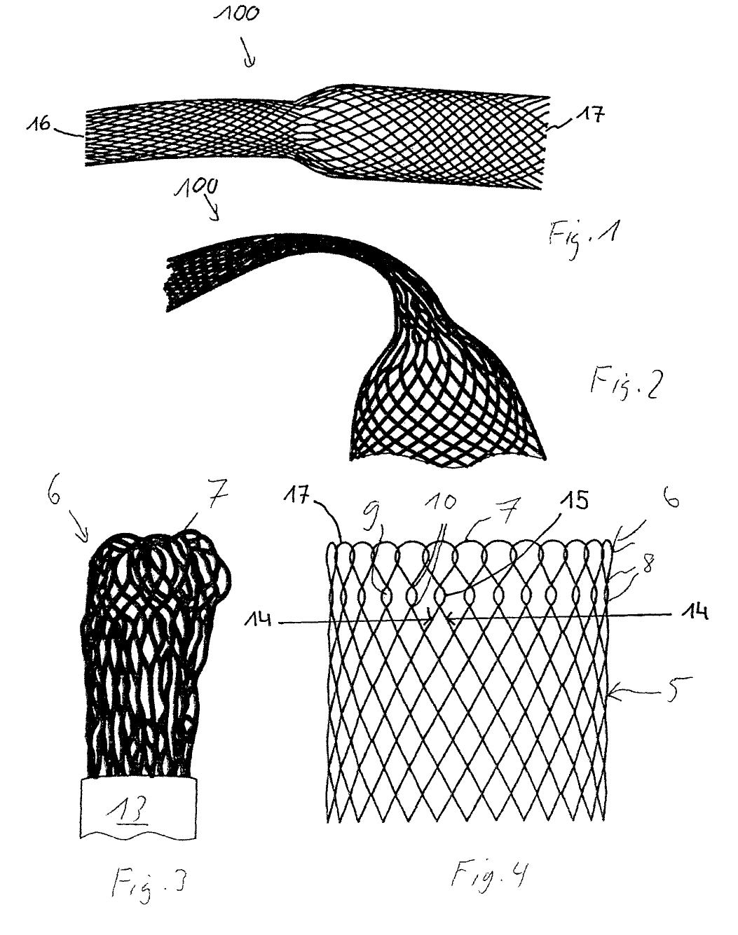

DE 2141252 A1 discloses a nasal inset for supporting the nasal cavity which has an approximately pear-shaped form and is made from an elastically deformable plastic basket. The approximate length of this nasal inset corresponds to approximately the depth of the nasal cavity of the user and is to be estimated according to FIG. 2. This shows a basket which is inserted into the nasal cavity through a nasal opening.

In WO 03/992765 A2 a self-expanding stent for insertion into a nasal passage for treatment of apnea is disclosed (FIG. 5). This stent is designed in such a way that it extends into the nasopharynx of the user. The stent is positioned between the uvula and the back wall of the pharynx. In order to keep the pharynx open, this stent inevitably must have a relatively large diameter. A distal end of this stent can have a tapered, approximately conically shaped section. Furthermore, the stent can be formed approximately cylindrical. Moreover, the stent can be enlarged at its proximal end in order to prevent movement of the proximal section into the nasal cavity.

DE 20 2009 010 388 U1 and PCT/EP 2010/004687, resp., disclose a fixation apparatus for fixing such an apnea stent in the airway whereas the fixation apparatus comprises two clamping elements between which the proximal end of the apnea stent can be fixed. Furthermore, an insertion tube for insertion of the apnea stent is disclosed which is bent at its distal end in order to facilitate insertion into the airway.

The apnea stent comprises a connection or coupling element at its proximal end so that an insertion rod can be coupled to the proximal end of the apnea stent. With the insertion rod the stent can be inserted into the insertion tube 6 and be compressed therein by inserting the insertion rod into the insertion tube so that the apnea stent is pulled into the insertion tube by the insertion rod.

US 2009/0010991 A1 discloses an expandable nasal stent which can be permanently implanted in the nasal entry or the nasal passage, resp. This stent is formed from a plastic or steel grid and provided with a filter element which is positioned in the proximal end of the stent. The stent can be coated with a drug.

US 2010/0211181 A1 and U.S. Pat. No. 5,336,163 disclose further nasal stents which are formed as filters in order to filter the inhaled air.

US 2010/0106255 A1 disclose a self-expanding stent which can be anchored with one end in the sinuses.

US 2003/0040772 A1 discloses a stent which comprises several shape-memory wires which are interwoven. According to this stent it is foreseen that the wires are being bent in order to create bent sections, whereas the bent sections are arranged in such a way that they define one end of the body. Furthermore, the wires are woven in such a way that they cross each other in order to obtain several angles, whereas at least one is an obtuse angle. The value for at least one obtuse angle can be increased by axial compression of the body. Such a stent can have loops at its ends, whereas the sections of the wire with which the loop is formed must not enlace each other and thereby are not twisted.

DE 10 2009 056 449 A1 discloses a medical apparatus which comprises a tubular latticework made from wire elements. At a distal end of the latticework, end meshes are created by the wire elements, whereas a first wire element of a first end mesh forms a loop with a tip which is arranged together with other loop tips on one single circumferential line axially delineating the latticework. It is foreseen that at least a second wire element of a second end mesh creates a loop by a double enlacement by 720° which in axial direction protrudes over the circumferential line of the loop tip. In the region of this loop an X-ray-marker element is provided.

US 2005/0283962 A1 discloses a procedure for braiding of a medical implant. It is foreseen that the medical implant is formed from a single wire, whereas at one or both end sections of the medical implant sections of the wire are formed to create a loop, whereas the sections of the wire with which the loop is closed do not enlace and, therefore, are not twisted.

US 2008/0221670 A1 discloses a radiopaque stent. The stent is formed by a wire braid, whereby at a distal end of the stent loops are provided, whereas the sections of the wire with which the loops are closed do not enlace and, therefore, are not twisted. Moreover, it is foreseen that sections of the loop extending in axial direction are interconnected by welding.

EP 1 722 695 B1 discloses an apparatus for recanalization of an at least partially occluded cavity. The apparatus comprises at least one compressible and self-expanding stent which is insertable into the region of the occluded cavity by means of a catheter.

Object of the present invention is to provide a compressible and self-expandable stent for splinting and/or keeping open a cavity, an organ duct, and/or a vessel in the human or animal body which simply and reliably expands.

Furthermore, it is an object of the present invention to provide a stent for splinting of the pharynx or of a nasal passage which during use, especially during insertion, does not create an uncomfortable feeling in the throat and on the mucosa, resp., or in the nasal passage.

It is a further object of the present invention to avoid that a section of the stent in its expanded state, inserted into the pharynx, kinks.

These objects are achieved by a stent comprising the features of claims 1 and 7 as well as by an apparatus comprising the features of claim 11. Advantageous embodiments are indicated in the sub-claims.

According to the invention a compressible, self-expandable stent for splinting and/or keeping open a cavity, an organ duct, and/or a vessel in the human or animal body is provided. The stent has a tubular shape and comprises a braid made from at least one wire. Sections of the wire or wires, resp., which extend between a proximal and a distal end of the stent are called strands. At the distal end of the stent each two strands are connected to each other with a connection element. Each of the connection elements is bent to form a loop, whereas each loop is formed by enlacement of the respective two strands to create a twist with which the loop is closed.

A compressible and self-expandable stent for splinting and/or keeping open a cavity, an organ duct, and/or a vessel in the human or animal body is provided especially as a stent for splinting of the pharynx or for splinting of a nasal passage.

A stent according to WO 2007/065408 comprises bendings at its distal end. These bendings are called round ends. The disadvantage in this embodiment of a stent is that the distal end of the stent during release from the insertion tube, i.e. immediately (ca. 0.5 to 1.5 cm) after the distal end of the stent is set free from the insertion tube, widens in a strongly conical or funnel-type shape. The result is that the distal end of the stent in a small section presses against the mucosa with a rather high pressure in that area where the first contact between the mucosa and the stent occurs, and in the upper pharynx region, resp. This pressure leads to a strong reflex which numerous patients sense as a gag reflex. This reduces tolerance and acceptance for such an embodied stent. In this context it is not relevant if the round ends extend approximately in the longitudinal direction or if these are bent convexly inwards.

A too strong conical widening of the distal end of the stent during expansion or during release from an insertion tube, resp., is prevented in the compressible, self-expandable stent for splinting of the pharynx according to the invention as it comprises at the distal end of the stent each a section of the respective wire bent to form a loop, whereas the section of the wire is enlaced to form a twist, with which the loop is closed.

The wire or the multiplicity of the wires may extend in one axial direction of the stent from proximal to distal or vice versa. The section of a wire extending between a proximal end of the stent and a distal end of the stent is called a strand. Each wire at least comprises two strands. A single wire may also be formed with a multiplicity of strands, the number of which preferably is even, as at the distal end none of the wires forms a free end. At the distal end of the stent two strands are connected by a connection section. It may especially be foreseen that the stent at its distal end for each of its connection sections comprises a loop. The term “for each of its connection sections” in the context of this invention means that almost all or most of the connection sections, resp., comprise a loop. If a few connection sections, e.g. max. 10% or max. 20% of the connection sections, do not comprise loops according to this invention, then the stent still widen cylindrically. On the contrast, a stent according to DE 10 2009 056 449 A1 only comprises one loop, i.e. not each connection section comprises a loop.

Surprisingly, it has turned out that by providing the loops with a twist, a distal section of the stent during expansion widens approximately parallel or cylindrical, resp., but not conically or funnel-shaped, resp., such as it is known from the stents known in the state-of-the-art. Further, the diameter of the distal section of a stent according to the present invention is smaller during retraction of an insertion tube, when the same length of the stent has been released, than for a funnel-shaped expanding stent according to the state-of-the-art.

In the region of the twist the strands are not additionally fixed with a clamp, a solder joint, or other types of connections so that the strands in the region of the twist can diverge a little bit during compression of the stent.

In this way pressing of the distal end of the stent into the mucosa is prevented as the first direct contact of the stent braid with the surrounding mucosa occurs only when the cylindrically opening stent braid in a major part has reached the diameter of the unloaded state. In this state the radial force of the braid is equally distributed over a larger area and not transmitted to the mucosa over a small section because no stiffened circular region at the distal end of the stent is created in the braid as it may occur with the inwards bent distal ends.

In this way the wearing comfort and especially insertion are improved for the patient. Consequently, tolerance and acceptance by patients increases without compromising functional efficiency.

The twist positioned next to the loop can be formed as a half stitch, whereas the half stitch is an enlacement of the wire by 180°.

During testing it was recognized that these effects do not occur when the twist is formed at least as a full stitch or more, whereas this corresponds to an enlacement of the wire of 360° (FIG. 6).

The effects described above also do not occur if at a distal end of the stent a section of the wire is formed to a loop, whereas the section of the wire, with which the loop is closed, is not twisted (FIG. 7). In such an embodiment during release of the stent from the tube a strong funnel-shaped expansion occurs and, furthermore, during insertion into an insertion tube the loops finally turn radially outwards, and an even more uncomfortable feeling is created for the user due to injury at the mucosa.

Examples for loops without twist are to be found, in addition to FIG. 7, also in the patent documents US 2003/0040772 A1, US 2005/0283962 A1 and US 2008/0221670 A1. Accordingly, a loop, in which a region of the wire is bent to form a loop, whereas in the section of the wire in which the loop is closed the wires touch each other only in the crossing point but do now enlace, is not a twist in the meaning of the present invention.

During expansion of the stent several effects play a role whereas it could not yet finally be determined of which importance the individual effects are for the described outcome. Yet, it is clear that the twist formed as a half stitch, whereas the half stitch is an enlacement of the wire by 180°, is the most favourable embodiment, and that an enlacement of the wire by a full stitch, which is an enlacement of the wire by 360°, is also applicable.

It turned out that during expansion of the stent the sections of the wires closely spaced in the region of the twist rub against each other. This friction acts against an opening of the stent in the distal end region. Therewith expansion in the distal end region is delayed so that no funnel or only a funnel with a very low opening angle results. In case of an enlacement of the wire with a half stitch the sections of the wire in the twisted region are laterally rather flexibly movable so that over a wide movement range a uniform frictional resistance prevails.

It is also expected that the turning or twisting, resp., during creation of the loop a torsional friction is induced in the regions neighbouring the wound section of the wire, which during expansion of the stent counteracts the expansion force and this torsional force in the wire counteracts the expansion force and, thereby, a too strong radial expansion is prevented, too.

The twisting of the sections of the wire, when creating the loop, leads to a local weakening of the material and to a constriction of the wire, resp. This constriction of the wire results in a higher flexibility. A tension in the distal end region of the stent, each between two wire sections which each are connected to each other by a loop, is induced in a stent, when compressed in an insertion tube, as the individual wire sections of the braid are compressed and arranged approximately in parallel to each other. Because of the flexible section in the region of the constriction the two wire sections can be bent towards each other with reduced force resulting in lower tensions. A low tension in the distal end section of the stent accomplishes a less strong expansion.

It is anticipated that the above explained effects lead to a less strong funnel-shaped expansion in the embodiment according to the invention.

Thereby, a compressible, self-expandable stent for splinting and/or keeping open a cavity, an organ duct, and/or a vessel in the human or animal according to the invention is provided. It can especially be applied for treatment of obstructive sleep apnea (OSA) and snoring. It is advantageous that—as the stent has to be applied by the patient himself each evening before sleep and has to be removed again in the morning—this procedure should be as painless as possible, i.e. can be executed without major irritation of the respective body tissue. Based on the effects as described above, i.e. the cylindrically-shaped expansion, this is possible with the stent according to the present invention.

In unloaded state of the stent the section of the wire which is enlaced to form a twist or in which the section of the wires closely spaced rub against each other, resp., can be widened in a way that in the region of the twist an elliptic section may be formed.

The stent may comprise a distal section, a proximal section and a transition section formed between these two sections, whereas the distal section has a higher widening than the proximal section.

Preferably, the loops at the distal end may by angled by 40° to 55° and especially by 45° towards the axial direction and conically tapered inwards so that the loops form a conically tapered distal end region of the stent.

Furthermore, in a distal region of the distal section, neighbouring the region of the loops, the braid may be formed tapered in the distal direction. The angle of this conical or tapered, resp., region towards the axial direction is 2° to 20°, or 15°, or 10°, and especially approximately 5° to 7.5°, resp.

The stent may also be formed in tubular shape with approximately constant diameter.

In both cases the stent is elastically deformable and nestles to the inner surface of the throat.

Further, according to the present invention a compressible, self-expandable stent for splinting and/or keeping open a cavity, an organ duct, and/or a vessel in the human or animal body, especially the pharynx is provided. The stent is formed by at least one wire and comprises a distal section, a proximal section, and a transition section located between the former two sections, whereas the distal section has a higher widening than the proximal section. The sections comprise a braid. In the region of the proximal section next to the transition section a decoupling region is formed in which each at least two sections of the at least one wire or the wires, resp., are twisted with each other.

In case of a stent embodied according to WO 2007/065408 a transition phase is comprised in which each at least two sections of the at least one wire are twisted with each other. Such a stent is shown in expanded, non-inserted state in FIG. 1. A disadvantage of such a stent is that the braid of the stent in the region neighbouring the twist frequently kinks or collapses, resp., in inserted state, at the transition from the nasal passage into the pharynx (nasopharynx) due to the bending (FIG. 2). A result is that in this bent region a rather broad, flat and, therefore, stiff region is formed which presses against the mucosa of the upper back wall and upper horizontal wall of the nasopharynx. As this region is a quite sensitive one this property reduces acceptance of such a stent by patients.

As in the region of the proximal section neighbouring the transition section a decoupling region is formed, in which each at least two sections of the at least one wire or the wires, resp., are twisted together, a collapse or a compression, resp., of the transition section in the bent state when inserted into the nasal passage is prevented. Due to the decoupling region even a local widening in the decoupling region is achieved so that the diameter of the transition section in expanded, bent state is approximately that of the proximal section and, therefore, the transition section is widened and does not maintain its funnel-type shape.

In that way a region with approximately constant diameter of the braid, spanning from the decoupling region in distal direction, is formed, whereas this region has an approximately constant radial force. Further, in the outer region nestling to the mucosa a highly uniform bending is achieved. The braid in this region does not press with a small section stronger against the mucosa than neighbouring sections. In this way opening of the stent is almost not recognized anymore.

Twisting of two sections may at least be formed by a full stitch, whereas the full stitch is an enlacement or twisting, resp., of the two sections of the wire by 360°. Preferably at least 1½, or at least 2, or at least 2½, or at least 3 stitches are provided.

The braid may have a multiplicity of openings, whereas the openings in the transition section are larger than the other openings in the proximal section. Thereby, alignment of the radial force in the transition section in bent state to that of the distal section is achieved and the braid in the transition section takes a widened shape in the expanded bent state.

The widened region of the braid in the distal section should have diamonds of approximately the same size in order to achieve an even pressure against the mucosa. In the transition section the diamonds should be significantly larger as otherwise the radial force would significantly increase at this position and become uncomfortably high for the patient.

The twisted decoupling region serves for functional separation of the distal widened section with high radial force and the proximal longer section with lower radial force. The twist prevents that forces originating from the distal section of the braid can be transmitted in an uncontrolled manner into the distal section of the braid or vice versa and that, consequently, unwanted displacements in the braid result which impair the functionality of the stent.

An even bending in the region of the mucosa at the upper back and the upper horizontal wall of the nasopharynx is thus achieved by providing the twist or the decoupling region, resp., in the region of the proximal section (with the smaller diameter) neighbouring the transition section. The distance of the twist in the proximal section from the transition section in proximal direction is at least ca. 0.2 to 2.5 cm and preferably at least ca. 0.5 cm to 1.5 cm.

Further, according to the present invention an apparatus for splinting and/or keeping open the pharynx with at least one expandable stent being compressible in an insertion tube is provided.

One section of the insertion tube may be bent in a circular segment shape in order to facilitate insertion into the throat. The circular segment shape bent section has a bending of approximately 80° to 120°.

The following descriptions apply to the above described devices according to the invention as far as applicable.

The stent may be braided from wire, fibers and/or filaments which cross each other. In the following only the term wire will be used whereas with this term also fibers and filaments are meant as long as the wire is not further defined.

The wire from which the stent is braided especially is a metal wire, preferably a nitinol wire or a steel wire.

The stent is elastically deformable and nestles to the inner surface of the nasal passage. The force transmitted from the stent to the nasal passage distributes evenly. This leads to no pain, reflexes or uncomfortable feeling caused by the stent in the sensitive throat. As the stent is braided it nestles to varying shapes of the throat and it is not necessary to individually adapt the stent to the throat of a given user.

With a few standard sizes of the stent which mainly differ in the length and the diameter of the tubular stent and its distal, its proximal sections and the transition section positioned between the former two sections, preferably with a single standard size, a stent fitting to almost any person can be provided.

The invention will now be exemplified in more detail on the basis of the embodiments illustrated in the drawings.

The drawings show in:

FIG. 1 a schematic side view of a compressible, self-expandable stent for splinting of a nasal passage according to the state-of-the-art in the non-expanded, non-bent state,

FIG. 2 the stent of FIG. 1 in the state when being inserted into the body and bent,

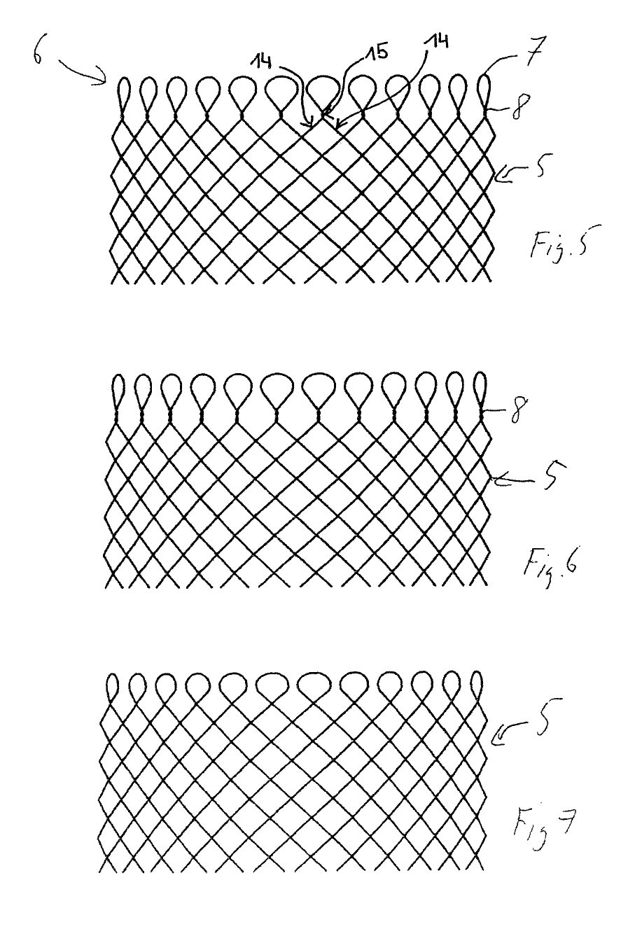

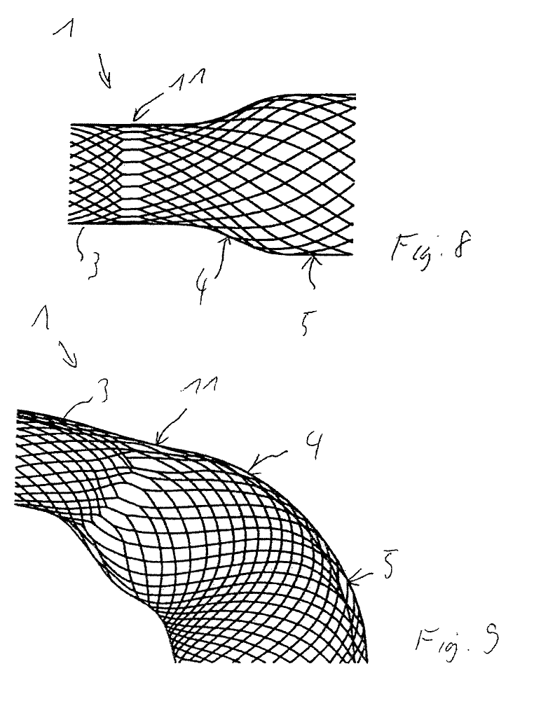

FIG. 3 a schematic side view of a distal end region of a non-expanded stent according to the present invention which is fully inserted into an insertion tube except of this distal end region,

FIG. 4 a schematic side view of a distal section of a stent according to the present invention in not fully expanded state,

FIG. 5 the distal section of a stent according to the present invention from FIG. 4 in fully expanded state,

FIG. 6 a schematic side view of a distal section of a stent, wherein loops are formed by multiple twisting,

FIG. 7 a schematic side view of a distal section of a stent, wherein loops are formed without twisting,

FIG. 8 a schematic side view of a stent according to the present invention, with proximal section, decoupling region, transition section, and distal section

FIG. 9 the stent from FIG. 8 in expanded, bent state or in state when inserted into the body, resp., and

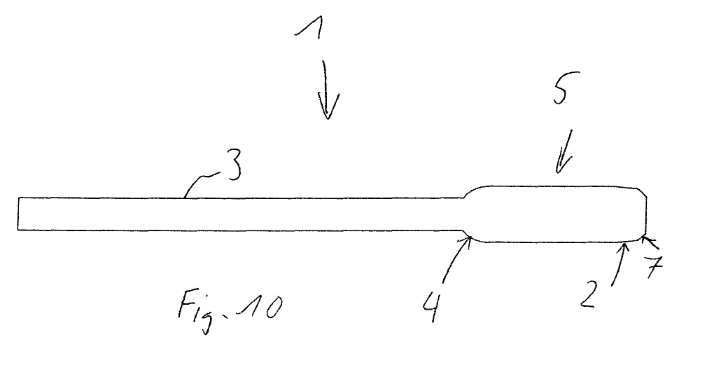

FIG. 10 a schematic side view of a stent according to the present invention with all sections.

In the following a compressible, self-expandable stent 1 for splinting of the throat and especially a distal end according to the present invention for a compressible, self-expandable stent 1 for splinting of the throat is described.

The stent 1 may be formed, if nothing else is described, similar to the stents according to the embodiments in WO 2007/065408 (FIG. 1) which are hereby incorporated by reference.

The stent 1 for splinting of the throat is formed with three-phases and provides a proximal section 3, a transition section 4 and a distal section 5 (FIG. 10). The proximal section 3 is formed approximately tubular with a constant diameter, whereas the wire is encapsulated in its distal end region. This region forms a type of fixation section. The transition section 4 is formed conically widening in distal direction. The distal section 5 is formed in tubular shape with approximately constant diameter.

Alternatively the stent may comprise a support body especially for splinting of a nasal passage which is formed approximately tubular with a constant diameter, whereas a fixation section is conically tapered (not shown) from its distal end towards the proximal end. The support body as well as the fixation section are integrally formed from one nitinol braid.

The stent 1 may be braided from a single wire or from a multiplicity of wires. The ends of the wire or the wires, resp., are encapsulated at the proximal end of the stent, or at the proximal end of the fixation section, or of the proximal section, resp., with a pencil-shaped or a cylinder-shaped, resp., body (not shown). The stent is made from an elastic wire with a diameter of 0.001 to 2 mm, especially a diameter of 0.05 to 0.5 mm and preferred with a diameter of 0.07 to 0.2 mm, mostly preferred 0.10 to 0.15 mm.

The wire or the multiplicity of wires extend in one axial direction of the stent 1 from proximal to distal and vice versa. The section of a wire which extends between a proximal end 16 of the stent and a distal end 17 of the stent is called a strand 14. Each wire comprises at least two strands 14. A single wire may also comprise several strands 14 the number of which preferably is even as at the distal end none of the wires forms a free end. At the distal end of the stent 1 two strands 14 are interconnected by a connection section 15. Each connection section 15 is bent to form a loop 7, whereas each loop 7 is closed by enlacing of each two strands 14 to create a twist 8 with which the loop 7 is closed.

According to the present invention at the distal end 6 of the stent 1 each one section of the wire is bent to form a loop 7 (FIGS. 3 to 5). This section of the wire is enlaced to form a twist 8 with which the loop 7 is closed. In this way each individual wire extending towards the distal end of the stent 1 at the distal end is led back to the proximal end of the stent. Concluding, at the distal end of the stent a multiplicity of loops are arranged radially circulating.

This means that preferably each section of the wire or each bent section of a wire, i.e. each connection section 15 which is formed at the distal end of the stent, comprises a loop 7.

Thus, the stent 1 comprises at its distal end radially circulating loops 7, spaced at equal distance from one another. The loops 7 in the non-expanded state are arranged approximately fan-shaped or anthoid, resp., cylindrically overlapping each other in a manner that no sections of the loops 7 protrude.

The radius of the loops 7 at the distal end in the expanded state of the stent 1 is in the range of ca. 0.5 mm to 2.0 mm, or from 0.7 mm to 1.0 mm. Thereby, a distal end 6 is provided with no individual free wire ends, whereas the loops and the twist 8 especially prevent a widening of the distal end 6 during expansion of the stent when retracting the insertion tube. The twisted loops 7 avoid irritation of the mucosa in the throat by the distal end 6 of the stent, as especially due to the loops with twist a distal section of the stent during expansion widens approximately parallel or cylindrically, resp., and not conically.

The twist 8 preferably comprises a half stitch, whereas the half stitch is an enlacement of the wire by 180°.

In non-expanded state of the stent the section of the wire which is enlaced to form a twist 8 is widened in such a way that in the region of the twist an elliptically widened section 9 is formed.

In the elliptically widened section the wire forms two friction regions 10 in which each two sections of the wire make contact.

The loop 7 or the loops 7 and the twist 8 may extend approximately in longitudinal direction or have conically tapered shape. In the case that the loop 7 or the loops 7 and the twist 8 are formed conically tapered the angle between the loop and the longitudinal direction is approximately 40° to 60° and preferably between 45° and 50°.

The length of the loops 7 in axial direction is ca. 2.5 mm to 3.5 mm and especially 3 mm in the state extending in the longitudinal direction, and 1.5 mm to 2.5 mm and especially 2 mm in the bent state.

In a distal region 2 of the distal section, in the region neighbouring the loops, the braid may be formed conically tapered. The angle of this conically tapered region towards the axial direction is 2° to 20°, or 15° or 10°, resp., and especially approximately 5° to 7.5°. The length of this distal region 2 is ca. 4-5 mm and especially 6 mm.

In a further embodiment of the stent according to the present invention which is described in the following the above described features can be provided in addition or alternatively.

In this further embodiment of the compressible, self-expandable stent according to the present invention comprising the proximal section 3, the transition section 4, and the distal section 5, a decoupling region 11 is formed in the region of the proximal section 3 neighbouring the transition section 4 (FIGS. 8, 9 and 10).

The decoupling region 11 is shifted backwards in the proximal section 3 in proximal direction relative to the transition section 4 by ca. 0.2 cm to 2.5 cm, preferably by 0.5 cm to 1.5 cm. Preferably the number of twists in the decoupling region is at least 1.5, or at least 2, or at least 2.5, or at least 3.

In the decoupling region individual strands run approximately in longitudinal direction of the stent 1 without crossing each other. Thus the decoupling region provides a high flexibility in radial direction. Thereby, it is ensured that over the entire length of the transition section and the distal section an approximately constant diameter is provided.

In the state when inserted into the body or into the throat, resp., and expanded, a section of the stent which comprises a proximal region of the proximal section 3, the transition section 4, and a distal region of the distal section 5 is bent in a circular segment shape. This section then has a bending of approximately 80 ° to 120°.

The decoupling region ensures that in case of such a bending a distal region of the proximal section 3 or a proximal region of the transition section 4, resp., do not fold together or collapse, resp., but remain in a cylindrically widened or slightly ball-shaped widened, resp., state.

An even bending of the stent at the upper back and the upper horizontal wall of the nasopharynx is achieved by providing the twist or the decoupling region, resp., in the region of the proximal section (with the smaller diameter) neighbouring the transition section.

In the following further features of the stent 1 are explained.

The distal section 5 of the stent forms a functional part of the apnea stent 1. The distal section 5 has a tubular shape and can be widened in a manner that the airway in the throat is kept open. Its diameter in expanded state is approximately 12 to 25 mm, and preferably 15 mm to 18 mm. The distal section 5 has a length of ca. 30 to 60 mm, or of ca. 40 to 50 mm, resp.

The proximal section 3 has a length of ca. 60 mm to 150 mm, or of ca. 80 mm to 110 mm, resp.

A fixation section neighbouring the proximal section has a length of approximately 10 to 40 mm, whereas this section may be fully or partially led out of the nose. The stent can be fixed at the body of the user via the fixation section so that the stent cannot move from the nasal passage of the user towards the throat. For that it is necessary that the fixation section extends through the nostril to the outside, in order to be fixed there with an appropriate fixation element. The fixation section thus is provided for fixing the stent in the region of the nose and has a funnel-type shape, whereas it widens from a proximal to a distal end.

The transition section of the stent is provided for connecting the proximal section with the distal section. The transition section 3 has a length of ca. 4 mm to 15 mm, or ca. 7 mm to 9 mm, resp.

The total stent has a length of 10 cm to 35 cm, or of 12 cm to 20 cm, resp.

The distal, the proximal, and the transition section are provided as a braid in which the wires or fibers or filaments, resp., cross each other. In the following only the term wire is used, whereas with this term also fibers and filaments are meant.

The wire preferably has a diameter of 0.001 to 2 mm, especially a diameter of 0.05 to 0.5 mm, and preferably a diameter of 0.07 to 0.2 mm, most preferred of 0.10 to 0.15 mm.

The stent is made from a shape memory material. The shape memory material is a biocompatible shape memory material, especially a metal or a metal alloy, especially stainless steel, or nitinol, or another biocompatible material such as polymer or monofilament and/or multifilament and/or composite glass fibers.

A connection or coupling element (not shown) is provided at the end 12 of the apnea stent 1, which can be coupled with the connection or coupling element of an insertion rod (not shown). The connection element is provided at one end of the insertion rod. In the present embodiment the connection element of the insertion rod is provided as a bushing and the connection element of the stent 1 as a ball positively connectable into the bushing.

For insertion of the stent 1 through a nasal passage into the throat the stent 1 by means of the insertion rod is inserted into an insertion tube and compressed therein. For that the insertion rod is inserted into the insertion tube so that the stent 1 is pulled into the insertion tube by the insertion rod.

For application of this apparatus for treatment of snoring and/or sleep apnea the stent 1 is inserted into one of the two nostrils when compressed in the insertion tube, and then advanced into the velopharynx and positioned at its application position. By retracting the insertion tube the stent 1 is released and self-expands to its preset size. Thereby, collapse of the throat and obstruction of the airway is prevented. Thereby a freely flowing airstream and normal breathing are enabled. After correct positioning of the stent 1 in the airway the insertion rod is disconnected.

This stent 1 is secured or fixed, resp., by a fixation element (not shown) against too deep penetration into and slipping out of the airway. With this fixation element the correct insertion depth of the apnea stent can be set and the apnea stent 1 be secured against unintended slipping inwards into the airway.

The insertion tube is arranged over the apnea stent 1 during insertion in order to position the apnea stent in the airway. The insertion tube at its distal end preferably has a circular segment shaped bent section which extends approximately over an angle range of ca. 80° to 120° with a radius of ca. 3 cm to 7 cm. Preferably the insertion tube comprises a two-layer tube with an inner, sufficiently hard appropriate polymer, e.g. PEBAX with a shore hardness of 60 Shore D to 80 Shore D, and an outer sufficiently smooth polymer, e.g. polyurethane with a shore hardness of 25 Shore D to 60 Shore D. The hardness of the polymer has significant influence on an optimal or exact, resp., adaptation of the insertion tube to the nasal passages, and its steerability down into the velopharynx during the insertion process. At the distal end of the insertion tube an insertion tip is formed in one piece, the inner as well as the outer diameter of which correspond to those of the insertion tube, and the tip being flush with the tube. The insertion tip may be made from a flexible, significantly smoother material such as e.g. polyurethane with a Shore hardness of 25 Shore D to 45 Shore D. The insertion tube 6 may also be provided as a single piece. Such a single piece insertion tube may consist of a uniform material. It is also possible to manufacture such a single piece insertion tube by an extrusion process with a continuous material gradient. In such an insertion tube a transition region exists where the share of the one material decreases continuously and the share of the other material increases. The materials of such a tube are e.g. PEBAX72 in the region of the main body and PEBAX35 in the region of the insertion tip. Regarding the material selection the above disclosure applies for a two-piece insertion tube in the same way.

At the distal end of the insertion tip of the insertion tube an atraumatic tip, preferably a rounding, is provided. Such a rounding can be provided at the outer and/or the inner side. In this way injury of the vessel walls during insertion of the apnea stent 1 are prevented and insertion is facilitated, as the insertion tube can ideally follow the bendings of the airway, and because the insertion tip is very smooth and flexible. The outer diameter of the insertion tube is up to 5 mm, preferably approximately 4 mm, in order to enable comfortable and pain-free insertion for the user. The inner diameter of the insertion tube is up to 4 mm in order to provide sufficient lumen for intake of the apnea stent. Due to the bending of the insertion tube bumping in the throat is prevented and thereby an injury-free insertion is ensured.

Instead of a rounding the insertion tip may alternatively be provided with a chamfer, preferably with an angle of 30° to 60°, more preferred with an angle of 45°.

Such a rounded tip is more elaborate in production than a chamfer. Yet, the rounded tip is even more safe during insertion of the stent. Further, in case of a chamfer the resulting thin-walled end of the chamfer may harden when plasticizers escape from the polymer material over the rather large surface.

These shaped insertion tips represent atraumatic tips. The harder the material of the tip is, the smoother and rounder the shape should be.

All length specifications provided in this application are average values. Dependent on whether children or tall adults are to be treated these values may respectively vary.

### LIST OF REFERENCE NUMBER

1 stent
2 distal region
3 proximal section
4 transition section
5 distal section
6 distal end
7 loop
8 twist
9 elliptically widened section
10 friction region
11 decoupling region
12 proximal end
13 insertion tube
14 strand
15 connection section
16 proximal end
17 distal end
100 stent according to the state-of-the-art

1. A compressible, self-expandable stent for splinting and/or keeping open a cavity, an organ duct, and/or a vessel in the human or animal body, whereas the stent is formed in tubular shape from at least one wire and the stent comprises a braid,
whereas sections of the wire or the wires, which extend between a proximal and a distal end of the stent are called strands, and at the distal end of the stents each two strands are connected by a connection section,
wherein each connection section is bent to form a loop,
wherein each loop by enlacement of each two strands forms a twist with which the loop is closed, and wherein the braid is made of the strands crossing each other.

2. The compressible, self-expandable stent according to claim 1,
wherein each twist is formed as a half stitch,
whereas the half stitch is an enlacement of the wire by 180°.

3. The compressible, self-expandable stent according to claim 1,
wherein in non-expanded state of the stent the section of the wire which is enlaced to form a twist is widened so that in the region of the twist an elliptically widened section is formed.

4. The compressible, self-expandable stent according to claim 3,
wherein in the elliptically widened section the wire forms two friction regions in which each two sections of the wire make contact.

5. The compressible, self-expandable stent according to claim 1, wherein
the stent comprises a distal section, a proximal section and a transition section positioned between these sections,
wherein the distal section has a larger widening than the proximal section.

6. A compressible, self-expandable stent for splinting and/or keeping open a cavity, an organ duct, and/or a vessel in the human or animal body,
whereas the stent is formed from at least one wire and comprises a distal section, a proximal section, and a transition section positioned between these sections,
whereas the distal section has a larger widening than the proximal section and the sections comprise a braid,
whereas sections of the wire or the wires which extend between a proximal and a distal end of the stent are called strands,
whereas in the region of the proximal section next to the transition section a decoupling region is formed in which each at least two strands are twisted with each other and run approximately in longitudinal direction of the stent.

7. The compressible, self-expandable stent according to claim 6,
wherein the twist of two sections is formed as at least a full stitch,
whereas the full stitch is an enlacement of the two sections of the wire by 360°.

8. The compressible, self-expandable stent according to claim 6,
wherein the braid has a multiplicity of openings,
whereas the openings in the transition section are larger than the other openings in the proximal section,
whereas the transition section takes a widened shape in the expanded bent state.

9. The compressible, self-expandable stent according to claim 6, wherein
the stent comprises a distal end formed such that each two strands are connected by a connection section, wherein each connection section is bent to form a loop, whereas each loop by enlacement of each two strands forms a twist with which the loop is closed.

10. An apparatus for splinting and/or keeping open the throat with at least one stent compressible in an insertion tube and being self-expandable, whereas the stent is a stent according to claim 1.

11. Apparatus according to claim 10,
wherein one section of the insertion tube is bent in a circular segment shape in order to facilitate insertion through a nasal passage into the throat, and the section has a bending of approximately 80° to 120°.
